(19) United States
(12) Patent Application Publication
(10) Pub. No.: US 2010/0221736 A1
(43) Pub. Date: Sep. 2, 2010

(54) METHOD FOR PREDICTING SUSCEPTIBILITY TO A MENTAL DISORDER

(76) Inventors: Kim Q. Do Cuenod, La Tour de Peilz (CH); Michel Cuenod, La Tour de Peilz (CH); René Gysin, Spiez (CH)

Correspondence Address: HARNESS, DICKEY & PIERCE, P.L.C., P.O. BOX 8910, RESTON, VA, 20195, US

(21) Appl. No.: 12/733,912

(22) PCT Filed: Aug. 28, 2008
(86) PCT No.: PCT/IB2008/002227

§ 371 Date: Mar. 26, 2010

Related U.S. Application Data
(60) Provisional application No. 60/935,826, filed on Aug. 31, 2007.

(51) Int. Cl. C12Q 1/68

(52) U.S. Cl. 435/6

(57) ABSTRACT

This invention relates generally to the field of mental disorders and, in particular, to a method for predicting susceptibility to a mental disorder, or a mental associated disorder.

### FIELD OF THE INVENTION

This invention relates generally to the field of mental disorders and, in particular, to a method for predicting susceptibility to a mental disorder, or a mental associated disorder.

### BACKGROUND OF THE INVENTION

Schizophrenia is a complex multifactorial brain disorder with a genetic component. Increasing evidence has implicated oxidative stress and glutathione (GSH) deficits in this disease. Many genetic studies have shown an association of gene markers with schizophrenia, but evidence for related functional alterations is sparse [Owen et al, 2005]. Recent gene-expression analysis, genetic studies, as well as quantifications of brain glutathione (GSH) levels in vivo and on postmortem tissues led to the hypothesis that a dysregulation of the GSH metabolism could be involved into the pathogenesis of schizophrenia [Tosic et al, 2006]. GSH levels were reported to be reduced in cerebrospinal fluid (−27%) and in medial prefrontal cortex (−52%) of schizophrenia patients [Tosic et al, 2006]. Similarly, GSH levels were found to be decreased (−40%) in the caudate region of postmortem-brain tissue from schizophrenia patients, as compared to control subjects [Yao et al, 2006].

GSH plays a crucial role as a cellular antioxidant scavenger of reactive oxygen species. It functions in maintaining intracellular redox potential, in detoxifying xenobiotics, in protecting cells from oxidative stress and cell death [Soltaninassab et al, 2000]. Cellular GSH levels are highly regulated and several substances known to produce an oxidative stress have been shown to increase GSH synthesis [Satoh et al, 2006].

GSH is synthesized in two consecutive enzymatic reactions: the first is catalyzed by the enzyme glutamate cysteine ligase (GCL) and the second by the glutathione synthetase (GSS). GCL consists of a catalytic (GCLC) and a modifier subunit (GCLM) [Meister, 1995].

Recently, International Patent Application WO 2005/068649 (Novartis) described a method for diagnosis of a mental disorder comprising determining the level of expression of at least one gene involved in regulating the intracellular glutathione (GSH) level and determining the level of activity of at least one protein involved in regulating the GSH level. The at least one protein involved in regulating the GSH comprises, GCLM or GCLC proteins. However this method determined the level of the GCLC protein which is involved in regulating the GSH level and no correlation has been made between a specific GCLC gene polymorphism and the susceptibility to develop mental disorder such as schizophrenia. Although this association was correlated with a significantly reduced GCLM expression in patients' fibroblasts, the functional implication of this gene on the disease remains unknown. In order to investigate for the presence of potential mutations in the GCLM gene, Applicants have screened the genomes of 72 Swiss and 281 Danish patients with schizophrenia. Ten sequence variations were identified, five of which were not previously described. None of these DNA changes was within the GCLM coding sequence and in silico analysis failed to reveal major functional impairment induced by these variations. Furthermore, a case-control analysis between the 72 Swiss patients and 83 Swiss controls revealed no significant association of any of these DNA variants with schizophrenia. Therefore, it is unlikely that functional mutations in the GCLM gene could play a major role in genetic predisposition to schizophrenia and further studies will be required to assess its etiological function in the disease.

It is commonly known from Walsh et al. 1996 and 2001 that a GCLC GAG trinucleotide repeat (TNR) polymorphism (TNR 7, 8 and 9) in the 5′ untranslated region of the gene is associated with variations in GSH levels.

Despite the disclosure of the foregoing patent application and scientific articles, there still remains a need to develop a reliable method for predicting susceptibility to a mental disorder, or a mental associated disorder, based on the determination of a direct correlation between a genetic polymorphism in a gene and said disorders.

This object has been achieved by providing a method for predicting susceptibility to a mental disorder, or a mental associated disorder, in a subject comprising obtaining a biological sample from said subject and, determining at least the presence of the GAG TNR polymorphism in the 5′-untranslated region of the GCLC gene in said biological sample, assessing whether the subject possesses a protective or a risk genotype associated with the presence of GAG TNR polymorphism in the 5′-untranslated region of the GCLC gene, thereby determining whether the subject is susceptible to develop a mental disorder, or a mental associated disorder.

### SUMMARY OF THE INVENTION

The present invention concerns a method for predicting susceptibility to a mental disorder, or a mental associated disorder.

A further object of the present invention is to provide a kit for predicting susceptibility to a mental disorder, or a mental associated disorder, in a subject.

Still another object of the invention is to provide a prognostic composition for predicting susceptibility to a mental disorder, or a mental associated disorder.

### DESCRIPTION OF THE FIGURES

FIG. 1. GCL activity and GCLC protein expression in schizophrenia patients and control subjects under untreated and t-BHQ treated conditions. A) GCL activity was measured in total cell extracts of cultured skin fibroblasts. The plot shows quantification of GCL activity of 25 patients and 25 controls subjects, expressed as nmol GSH synthesized per min and per mg protein. Each box describes 25 and 75% values, the horizontal line inside the box depicts median numbers, the whisker bars show the values in the 1.5 box lengths range, the open circle depicts an outlier value. Note that GCL activity in t-BHQ treated fibroblasts of patients was significantly lower than in controls. B) The plot shows GCLC protein levels of 26 patients and 26 controls subjects, determined as α-Tubulin corrected arbitrary units. Note that GCLC protein expression of patients was significantly lower than in controls under both conditions. ** P<0.01 *** P<0.001 versus the respective controls were calculated using ANOVA test (two-tailed). C) Representative Western Blots of GCLC, GCLM and α-Tubulin of two controls and two patients under untreated and t-BHQ treated conditions.

FIG. 2. Different regulation of GSH synthesis in patients and controls. The plots show GCLM and GCLC protein expression under untreated (gray diamonds) and t-BHQ treated (black triangles) conditions of A) 26 controls subjects and B) 26 schizophrenia patients. Note that the correlation between GCLM and GCLC is lost in patients. GCLC protein expressions compared with GCL activities under untreated (gray diamonds) and t-BHQ treated (black triangles) conditions of C) 24 controls subjects and D) 24 schizophrenia patients. Note that in patients under oxidative stress, the GCL activity is dependant on GCLC protein levels. Ratios of GCLM vs. GCLC protein amounts under untreated conditions (gray squares) and after t-BHQ treatment (black triangles) compared with GCL activities of E) 24 controls subjects and F) 24 schizophrenia patients. Note that in patients, GCL activity is inversely correlated with the GCLM/GCLC ratio. R is the Spearman correlation, P is the probability of type I error.

FIG. 3. Evidence for a functional relevance of the GCLC GAG TNR polymorphism in the Swiss cohort. As genotypes 7/7 & 7/9 were present more often in control subjects, while genotypes 7/8, 8/8, 8/9 & 9/9 were present more often in patients, we regrouped the data of GCL activity, GCLC protein expression and GSH content of all tested subjects according their genotypes (7/7 & 7/9 vs. 7/8, 8/8, 8/9 & 9/9) and we compared the groups. The plots show (A) GCL activity (t-BHQ), and (B) GCLC protein expression (t-BHQ) of 38 subjects with genotypes 7/7 & 7/9 and 11 subjects with genotypes 7/8, 8/8, 8/9 & 9/9. C) The plot shows GSH content (baseline) of 56 subjects with genotypes 7/7 & 7/9 and 16 subjects with genotypes 7/8, 8/8, 8/9 & 9/9. Each box describes 25 and 75% values, the horizontal line inside the box depicts median numbers, the whisker bars show the values in the 1.5 box lengths range. * P<0.05 ** P<0.01 versus the respective controls was calculated using Mann-Whitney U test (two-tailed).

### DETAILED DESCRIPTION OF THE INVENTION

The present invention relates to a method for predicting susceptibility to a mental disorder, or a mental associated disorder, in a subject comprising

i) obtaining a biological sample from said subject and,

ii) determining at least the presence of GAG trinucleotide repeat (TNR) polymorphism in the 5′-untranslated region of the glutamate cystein ligase catalytic subunit (GCLC) gene in said biological sample,

iii) assessing whether the subject possesses a protective or a risk genotype associated with the presence of GAG trinucleotide repeat (TNR) polymorphism in the 5′-untranslated region of the glutamate cystein ligase catalytic subunit (GCLC) gene, thereby determining whether the subject is susceptible to develop a mental disorder, or a mental associated disorder.

The term “comprise” is generally used in the sense of include, that is to say permitting the presence of one or more features or components.

A “mental disorder” is a clinically significant psychological pattern that occurs in a subject and is usually associated with distress or disability that is not expected as part of normal development or culture. Definitions, assessments, and classifications of mental disorders can vary, but guideline criterion listed in the 4^{th} edition of DSM (Diagnostic and Statistical Manual of Mental Disorders, Francis A Editor, American Psychiatric Press, Wash. D.C., 1994) are widely accepted by mental health professionals. Preferably, the mental disorder is selected from the group of schizophrenic disorders, affective disorders, psychoactive substance use disorders, personality disorders, delirium, dementia, epilepsy, panic disorder, obsessive compulsive disorder, intermittent explosive disorder, impulse control disorder, psychosis, attention-deficit-hyperactivity disorder (ADHD), and manic or psychotic depression and autism.

Usually, the mental disorder is selected from the group comprising schizophrenic disorders, affective disorders, psychoactive substance use disorders, personality disorders, delirium, dementia, epilepsy, panic disorder, obsessive compulsive disorder, intermittent explosive disorder, impulse control disorder, psychosis, attention-deficit-hyperactivity disorder (ADHD), and manic or psychotic depression and autism.

Preferably, the schizophrenic disorder is selected from the group comprising schizophrenia, schizophrenic form disorders or schizoaffective disorders.

Schizophrenic Disorders include Schizophrenia, Catatonic, Subchronic, (295.21); Schizophrenia, Catatonic, Chronic (295.22); Schizophrenia, Catatonic, Subchronic with Acute Exacerbation (295.23); Schizophrenia, Catatonic, Chronic with Acute Exacerbation (295.24); Schizophrenia, Catatonic, in Remission (295.55); Schizophrenia, Catatonic, Unspecified (295.20); Schizophrenia, Disorganized, Subchronic (295.11); Schizophrenia, Disorganized, Chronic (295.12); Schizophrenia, Disorganized, Subchronic with Acute Exacerbation (295.13); Schizophrenia, Disorganized, Chronic with Acute Exacerbation (295.14); Schizophrenia, Disorganized, in Remission (295.15); Schizophrenia, Disorganized, Unspecified (295.10); Schizophrenia, Paranoid, Subchronic (295.31); Schizophrenia, Paranoid, Chronic (295.32); Schizophrenia, Paranoid, Subchronic with Acute Exacerbation (295.33); Schizophrenia, Paranoid, Chronic with Acute Exacerbation (295.34); Schizophrenia, Paranoid, in Remission (295.35); Schizophrenia, Paranoid, Unspecified (295.30); Schizophrenia, Undifferentiated, Subchronic (295.91); Schizophrenia, Undifferentiated, Chronic (295.92); Schizophrenia, Undifferentiated, Subchronic with Acute Exacerbation (295.93); Schizophrenia, Undifferentiated, Chronic with Acute Exacerbation (295.94); Schizophrenia, Undifferentiated, in Remission (295.95); Schizophrenia, Undifferentiated, Unspecified (295.90); Schizophrenia, Residual, Subchronic (295.61); Schizophrenia, Residual, Chronic (295.62); Schizophrenia, Residual, Subchronic with Acute Exacerbation (295.63); Schizophrenia, Residual, Chronic with Acute Exacerbation (295.94); Schizophrenia, Residual, in Remission (295.65); Schizophrenia, Residual, Unspecified(295. 60); Delusional (Paranoid) Disorder (297.10); Brief Reactive Psychosis (298.80); Schizophreniform Disorder(295. 40); Schizoaffective Disorder (295.70); Induced Psychotic Disorder (297.30); Psychotic Disorder NOS (Atypical Psychosis) (298.90).

Affective Disorders include Major Depressive Disorder; Severe with Psychotic Features (296.33); Bipolar I Disorder, Single Manic Episode, Severe with Psychotic Features (296.23); Bipolar I Disorder, Most Recent Episode Hypomanic (296.43); Bipolar I Disorder, Most Recent Episode Manic, Severe with Psychotic Features (296.43);Bipolar I Disorder, Most Recent Episode Mixed, Severe with Psychotic Features (296.63);Bipolar I Disorder Most Recent Episode Depressed, Severe with Psychotic Features (296.53); Bipolar I Disorder, Most Recent Episode Unspecified (296.89); BipolarII Disorder (296.89); Cyclothymic Disorder (301.13); Bipolar Disorder NOS (366); Mood Disorder Due To (General Medical Condition) (293.83)

Mood Disorder NOS (296.90); Conduct Disorder, Solitary Aggressive Type (312.00); Conduct Disorder, Undifferentiated Type (312.90); Tourette's Disorder (307.23), Chronic Motor Or Vocal Tic Disorder (307.22); Transient Tic Disorder (307.21); Tic Disorder NOS (307.20).

### Psychoactive Substance Use Disorders

Alcohol Withdrawal Delirium (291.00); Alcohol Hallucinosis (291.30); Alcohol Dementia Associated with Alcoholism (291.20); Amphetamine or Similarly Acting Sympathomimetic Intoxication (305.70); Amphetamine or Similarly Acting Sympathomimetic Delirium (292.81); Amphetamine or Similarly Acting Sympathomimetic Delusional Disorder (292.11); Cannabis Delusional Disorder (292.11); Cocaine Intoxication (305.60); Cocaine Delirium (292.81); Cocaine Delusional Disorder (292.11); Hallucinogen Hallucinosis (305.30); HallucinogenDelusional Disorder (292.11); Hallucinogen Mood Disorder (292.84); Hallucinogen Post hallucinogen Perception Disorder (292.89); Phencyclidine (PCP) or Similarly Acting Arylcyclohexylamine Intoxication (305.90); Phencyclidine (PCP) or Similarly Acting Arylcyclohexylamine Delirium (292.81);Phencyclidine (PCP) or Similarly Acting Arylcyclohexylamine Delusional Disorder (292.11); Phencyclidine (PCP) or Similarly Acting Arylcyclohexylamine Mood Disorder (292.84);Phencyclidine (PCP) or Similarly Acting Arylcyclohexylamine Organic Mental Disorder NOS (292.90); Other or Unspecified Psychoactive Substance Intoxication (305.90); Other or Unspecified Psychoactive Substance Delirium (292.81); Other or Unspecified Psychoactive Substance Dementia (292.82); Other or Unspecified Psychoactive Substance Delusional Disorder (292.11); Other or Unspecified Psychoactive Substance Hallucinosis (292.12); Other or Unspecified Psychoactive Substance Mood Disorder (292.84); Other or Unspecified Psychoactive Substance Anxiety Disorder (292.89); Other or Unspecified Psychoactive Substance Personality Disorder (292.89); Other or Unspecified Psychoactive Substance Organic Mental Disorder NOS (292.90).

Delirium (293.00); Dementia (294.10); Obsessive Compulsive Disorder (300.30); Intermittent Explosive Disorder (312.34); and Impulse Control Disorder NOS (312.39).

Schizophrenia is a severe mental disorder characterized by a variety of signs and symptoms. However, no single symptom is definitive for diagnosis. Rather, diagnosis encompasses a pattern of signs and symptoms, in conjunction with impaired occupational or social functioning (DSM-IV).

According to the invention, the term “schizophrenia” is preferably used in the sense of, but not limited to, the criteria for diagnosing schizophrenia from the American Psychiatric Association's Diagnostic and Statistical Manual of Mental Disorders (DSM), in the most recent version DSM-IV. To be diagnosed as having schizophrenia, a person must display: (A) Characteristic symptoms: two or more of the following, each present for a significant portion of time during a one month period (or less, if successfully treated): delusions, hallucinations, disorganized speech (e. g. frequent derailment or incoherence), grossly disorganized or catatonic behavior, negative symptoms, i. e. affective flattening (lack or decline in emotional response), alogia (lack or decline in speech) or volition (lack or decline in motivation). Only one Criterion A symptom is required if delusions are bizarre or hallucinations consist of hearing voices.

(B) Social/Occupational dysfunction: for a significant portion of the time since the onset of the disturbance, one or more major areas of functioning such as work, interpersonal relations, or self-care are markedly below the level achieved prior to the onset.

(C) Duration: continuous signs of the disturbance persist for at least six months. The six month period must include at least one month of symptoms (or less, if successfully treated) that meet Criterion A.

The DSM-IV contains five sub-classifications of schizophrenia. These are catatonic type (where marked absences of peculiarities of movement are present), disorganised type (where thought disorder and flat or inappropriate affects are present together), paranoid type (where delusions and hallucinations are present but thought disorder, disorganised behaviour and affective flattening is absent), residual type (where positive symptoms are present at low intensity only) and undifferentiated type (psychotic symptoms are present but the criteria for paranoid, disorganised, or catatonic types have not been met).

Symptoms may also be described as “positive symptoms” (those additional to normal experience and behaviour) and negative symptoms (the lack or decline in normal experience or behavior).“Positive symptoms” describe psychosis and typically include delusions, hallucinations and thought disorder. “Negative symptoms” describe inappropriate or non present emotion, poverty of speech and lack of motivation.

The diagnosis of a mental disorder in a human being can be made based on the results of polymorphism/haplotype determination. The patient to be tested may have one or a plurality of polymorphisms and/or at least one combination of polymorphisms of at least one genomic copy which are associated with a mental disorder, preferably schizophrenia. If such a diagnosis is given, the patient is at risk of developing a mental disorder, preferably schizophrenia. In the other case, the subject possesses a protective genotype.

Preferably, the personality disorder is selected from the group comprising paranoid, schizoid, schizotype, antisocial and borderline disorders.

Usually, the “biological sample” of the invention is selected from the group comprising whole blood, semen, saliva, tears, urine, fecal material, sweat, buccal smears, skin, and biopsies of muscle, brain tissue, nerve tissue and hair.

Most preferably, the biological sample is blood or skin. More preferably, the biological sample is and one or more fibroblast cells extracted from the skin.

As used herein the term “polymorphism” refers to the variation that exists in the DNA sequence for a specific marker or gene. That is, by definition, the possibility that exist more than one allele for a gene or marker.

As used herein “susceptibility” refers to the risk, for the subject, of developing or suffering from a mental disorder, or a mental associated disorder.

“GAG trinucleotide repeat (TNR) polymorphism” refers to a trinucleotide repeat of DNA in a gene that contains the same trinucleotide sequence repeated many times. These repeats are a subset of unstable microsatellite repeats that occur throughout all genomic sequences.

In the present invention, the TNR is composed of a GAG repeat in the 5′-untranslated region of the glutamate cysteine ligase catalytic subunit (GCLC) gene. Usually, this GAG TNR polymorphism is located 10 base pairs upstream of the cDNA start codon.

GSH is synthesized in two consecutive enzymatic reactions: the first is catalyzed by the enzyme glutamate cysteine ligase (GCL) and the second by the glutathione synthetase (GSS). GCL consists of a catalytic (GCLC) and a modifier subunit (GCLM). In 1992, Gipp et al. cloned and sequenced the full-length GCLC cDNA encoding a 367-amino acid protein with a calculated molecular mass of approximately 73 kD. Fluorescence in situ hybridization (FISH) experiments localized the gene to 6p12 on the human chromosome.

Applicants have shown that schizophrenia, a mental disorder, is associated with a decreased capacity to synthesize GSH. In skin fibroblasts of schizophrenia patients and control subjects they investigated GSH synthesis in response to oxidative stress and compared the genotype distribution of the GAG TNR polymorphism in the GCLC gene in two independent cohorts. Applicants report evidence that schizophrenia patients have a decreased capacity to synthesize GSH, most likely of genetic origin.
The major findings can be summarized as follow:
a) GCL activity in patients, as compared to controls, was impaired in skin fibroblast cultures under conditions of oxidative stress.
b) This reduced GCL activity correlated with decreased GCLC protein expression that could not be compensated for by the observed increase in GCLM protein expression.
c) This reduced GCL activity correlated also with modified amino acid levels.
d) In a Swiss cohort patients had a different genotype distribution of the GAG TNR polymorphism in the GCLC gene as compared to controls. The disorder-associated genotypes of this polymorphism correlated with a decrease in GCLC protein expression, GCL activity and GSH content.
e) A second case-control study in a Danish cohort confirmed this difference in genotype distribution of the GCLC GAG TNR polymorphism and revealed a disorder-associated genotype 8/8 with an odds ratio of 2.96. Previous genetic and biochemical studies in five independent cohorts of patients have demonstrated an association between schizophrenia and parameters of the GSH metabolism [Tosic et al, 2006]. Surprisingly, the present results now provide genetic and functional evidence that a decreased capacity to synthesize GSH under conditions of oxidative stress contributes to the vulnerability/susceptibility for schizophrenia.

The human brain, being metabolically very active, is particularly sensitive to an impaired capacity to react against an oxidative stress [Sokoloff, 1999]. Some environmental risk factors for schizophrenia such as viral infections, inflammations, or obstetrical complications are known to increase oxidative stress [Robertson et al, 2006]. In addition, psychological stress can generate oxidative stress via the hypothalamic-pituitary-adrenal axis, especially in hormone sensitive or dopamine innervated brain regions [Piazza et al, 1996].

GCL activity under conditions of t-BHQ (tert-butylhydroquinone) treatment was lower in patients than in controls. The enzymatic activity of GCL can be influenced by many physiological parameters, including: post-translational modification of its subunits, the ratio of modifier vs. catalytic GCL subunit, as well as the concentrations of substrates, co-substrates and the product, GSH [Soltaninassab et al, 2000]. GCL activity was measured under saturating conditions of cysteine and ATP. Therefore, aside from post-translational modifications, the enzymatic activity depended primarily on the GCLM/GCLC ratio and on the total protein amounts of each of the two GCL subunits.

Surprisingly, Applicants' results showed that GCL activity in patients was limited by a decreased GCLC protein expression that could not be compensated for by an increased GCLM protein expression. As they earlier observed that GCLM and GSS gene expressions were decreased in schizophrenia in a coordinated manner [Tosic et al, 2006], they analyzed whether the decreased GCLC protein expression was due to a generally limited capacity of the cells to react against t-BHQ treatment, or whether the decreased GCLC protein expression was due to a specific polymorphism (or mutation) in the GCLC gene. Aside from GCLC and GCLM, the protein expressions of three further phase II genes were determined: NQO1, NQO2 and NRF2 (data not shown). GCLC was the only protein with an impaired expression in patients. This observation strengthened the hypothesis that decreased GCLC protein expression was due to a polymorphism (or mutation) in the GCLC gene.

Whole genome linkage analysis for complex disorders are generally performed with markers that are either present or absent on an allele, resulting in three possible genotypes. In contrast, the GAG TNR polymorphism in the 5′-untranslated region of the GCLC gene has either 7, 8 or 9 GAG repeats, resulting in six possible genotypes (7/7, 7/8, 7/9, 8/8, 8/9, & 9/9). Assuming that a marker used in a linkage study for schizophrenia would have been linked to the GCLC GAG TNR polymorphism, the possible association with the disorder would have been underestimated.

In a preceding study Applicants have measured GCLC mRNA levels in skin fibroblasts cultures of patients and controls [Tosic et al, 2006]. The study reported a trend (P=0.064) of lower GCLC mRNA levels in patients as compared to controls. In the present study, analyses between GCLC GAG TNR genotypes and GCLC mRNA levels showed no correlation. Average GCLC gene expression of genotypes 7/7 and 7/9 was 1.1±1.3; average gene expression of all other genotypes together was 1.2±0.8. The GAG TNR in the GCLC gene is just 10 base pairs upstream of the cDNA start codon and is expressed in the mRNA (http://www.ncbi.nlm.nih.gov/entrez/viewer.fcgi?db=nucleotide&val=45359851). The fact that, in contrast to GCLC gene expression, GCLC protein expression was decreased in the disorder-associated TNR genotypes suggests that the GAG TNR in the GCLC gene influences mRNA transport or translation rather than its expression. The GAG TNR polymorphisms in the GCLC gene were not sufficient to explain the GCL activity in all subjects. Aside from patients with disorder-associated genotypes (7/8, 8/8, 8/9 & 9/9) there are subjects with low GCL activity although they had the genotypes 7/7 or 7/9. Thus, it is conceivable that further polymorphisms (or mutations) in the GCLC and/or GCLM genes could be at the origin of low GCL activity. Moreover, a combination of various subtle alterations in different signalling pathways could also ultimately lead to decreased GSH synthesis.

As found in the preceding study on the same Danish cohort an association between genetic variants of GCLM and schizophrenia [Tosic et al, 2006], Applicants tested for possible interactions between the disorder-associated single nucleotide polymorphisms in the GCLM gene (rs2301022 and rs3170633) and the GAG TNR polymorphism of the GCLC gene. No significant interaction was observed (data not shown) pointing to independent contributions of the disorder-associated polymorphisms of the two GCL genes.

Aside from genetic association with schizophrenia the present results provide evidence for a functional relevance of the GCLC GAG TNR polymorphism for the first time in a non-tumor cell line.

In the Swiss cohort the two most frequent genotypes 7/7 and 7/9 were consistently associated with higher GCLC protein expression, GCL activity and GSH content, as compared to the other genotypes (7/8, 8/8, 8/9, & 9/9). The distribution of the three alleles in the controls of the Danish cohort (61% of allele 7, 16% of allele 8 & 23% of allele 9) was similar to the one described by Walsh and colleagues¹⁷ in a randomly selected white population of 100 individuals in New York (64% of allele 7, 15% of allele 8 & 21% of allele 9). In contrast to the randomly selected control subjects in our Danish and the New York cohorts, controls in the Swiss cohort were carefully selected according to the Diagnostic Interview for Genetic Studies (DIGS),18 excluding subjects with a history of a major psychiatric illness or having a first order relative with a history of psychosis. Both Swiss and Danish case-control studies showed significantly more 7/7 genotypes in control subjects, as compared to patients. The fact that all but one control subject in the Swiss cohort had a genotype containing at least one 7 allele suggests that subjects containing a genotype without a 7 allele have an increased vulnerability for schizophrenia. Compared to the average allele distribution in the randomly chosen control subjects of our Danish and the New York [Walsh et al, 2001] cohorts, the control group of the Swiss cohort had a strong shift towards genotypes 7/7, whereas patients in the Swiss and the Danish cohort had a shift towards genotypes 8/8 or 9/9. Moreover, the case-control study in the Danish cohort showed a strong association between schizophrenia and the genotype 8/8. The presence of more genotypes 8/9 in the controls of the Danish cohort, as compared to patients, could be due to the fact that subjects of families with a history of a major illness were not excluded.

The association between GCLC GAG TNR genotypes and GSH content in skin fibroblast cultures was different, compared to tumor cell lines as described by Walsh and colleagues [Walsh et al, 2001]. In both studies the genotype 8/8 was associated with low GSH content. In tumor cell lines allele 7 was associated with lower GSH content than alleles 8 and 9. In contrast, in skin fibroblast cultures genotypes 7/7 and 7/9 were associated with higher GSH contents than genotypes containing an 8 allele or genotype 9/9. The divergent associations observed in tumor cell lines and in skin fibroblast could have the following reasons. Tumor cell lines represent a heterogeneous group of cells with uncontrolled parameters that can influence GSH contents. Tumor cell lines have altered metabolisms and often altered GSH contents. Moreover, tumor cell lines originate from a variety of tissues, and it is known that GSH contents vary considerably among different cell types. In summary, non-tumor skin fibroblast cultures represent a more reliable model for comparisons between GCLC GAG TNR genotypes and GSH contents than a mix of 59 different tumor cell lines.

Therefore also encompassed in the present method of the invention is the method wherein the GAG TNR polymorphism in the 5′-untranslated region of the GCLC gene in said biological sample is associated with lower glutathione GSH content, and/or decreased level of GCLC protein and/or a lower capacity to synthesize GSH protein.

Mental disorders, and in particular schizophrenia, are complex disorders and not any single vulnerability factor alone would be sufficient for the development of these illnesses.¹ A combination of several genetic and environmental factors is required to promote vulnerability into the full-fledged appearance of the schizophrenia disorder [Mimics et al, 2006]. Genetic polymorphisms that cause a deficit in GSH synthesis were described to participate in a spectra of various disorders, as for example: increased susceptibility to oxidative stress [Yang et al, 2002], increased susceptibility for myocardial infarction [Koide et al, 2003], increased susceptibility for hemolytic anemia [Beutler et al, 1999], and in special cases increased susceptibility to neurological problems or mental retardation [Dahl et al, 1997].

Surprisingly, Applicants have also found that the GCLC GAG TNR polymorphism is associated with mental associated disorders such as for example bipolar disorder. Example 4 shows that in the Lausanne cohort a different GAG TNR distribution in bipolar disorder patients has been found in comparison to controls.

Accordingly, the present invention also envisions that the method is useful for predicting susceptibility to a mental associated disorder. As used herein “a mental associated disorder” is a disorder which is associated, comorbid and/or secondary, and/or belongs to the spectrum of mental disorders.

Usually, the associated disorder is selected from the group comprising a major depressive disorder, a bipolar disorder, a personality disorder, an obsessive compulsive disorder, autism, cardiovascular disorder and diabetes.

Preferably, the personality disorder is selected from the group comprising paranoid, schizoid schizotype, antisocial and borderline disorders.

Mouse gene knockout models have revealed that absence of GCLC is lethal.³¹ Genetic studies in humans have described two different mutations in the GCLC gene that were associated with hemolytic anemia and learning disabilities. Furthermore, these mutations were associated with increased susceptibility for myocardial infarction [Beutlert et al, 1999]. Similarly, a polymorphism in the promoter of GCLM was found to be associated with an increased susceptibility for myocardial infarction [Nakamura et al, 2002]. Interestingly, a greater risk for cardiovascular morbidity has been described for schizophrenia [Curkendall et al, 2004]. Mutations in GSS, the second enzyme for the GSH synthesis, were shown to be associated with 5-oxoprolinuria, hemolytic anemia, acidosis and variable neurological symptoms [Dahl et al, 1997].

The present method for predicting susceptibility to a mental disorder, or an associated disorder, further comprises the determination of at least one polymorphism in a second gene involved in an epistatic interaction with GCLC.

Complex phenotypes are not typically the result of variation of a single genetic locus, but rather the result of interplay between interactions among multiple genes and a variety of environmental exposures. These interactions are called epistatic interactions and they can be either synergistic (positive) or antagonistic (negative).

Preferably, the second gene involved in an epistatic interaction with GCLC is selected from the group of genes coding for Catechol O-methyltransferase, Dysbindin, Neuregulin-1, Protein kinase B, Disrupted in schizophrenia 1 protein, glutamic acid decarboxylase 1, Regulator of G-protein signaling 4, Receptor tyrosine-protein kinase erbB-4, Serine/threonine-protein phosphatase 2B catalytic subunit gamma isoform, Early growth response protein 3, Proline dehydrogenase 2, D-amino acid oxidase activator, Neuronal acetylcholine receptor subunit alpha-2, Glutamate-cysteine ligase modifier subunit, Glutathione synthetase, Glutathione peroxidase 1, Gamma-glutamyltranspeptidase 1, Nuclear respiratory factor 1, Nuclear factor erythroid 2-related factor or Nuclear factor erythroid 2-related factor 3.

Usually, traditional methods of analyzing genetic association as well as those described by Culverhouse et al, 2004 are known from the person skilled in the art and can be applied to analyze and predict these epistatic interactions.

Usually, the presence of GAG TNR polymorphism in the 5′-untranslated region of the GCLC gene in said biological sample is determined by Polymerase Chain Reaction and gel separation of the polymorphic fragments. However, all techniques useful for determining the presence of GAG TNR polymorphism may be routinely applied by the person skilled in the art.

Conventionally also, PCR-RFLP (Restriction Fragment Length Polymorphism) method is used for determining genetic polymorphism. The principle of this method involves the procedure of: (1) reacting a nucleotide sequence-specific restriction enzyme to PCR amplified product when a restriction enzyme specifically recognizing a polymorphic site does exist; (2) subjecting the product to electrophoresis; and (3) detecting the presence or absence of cleavage due to differences in the nucleotide sequences as a difference in molecular weight.

PCR-SSPC (Single-Strand Conformation Polymorphism) method has also been used for the detection of genetic polymorphism. When a DNA denatured to single strands is returned to nondenaturing conditions, it forms higher-order molecular structures, such as a hairpin loops. This intramolecular structure is greatly affected and altered by even a single nucleotide mutation. The PCR-SSPC method is based on the principle of detecting structural differences such as the differences in mobility in polyacrylamide gel electrophoresis under nondenaturing conditions.

Furthermore, genetic polymorphism is also analyzed using automatic fluorescence sequencer. According to this method, first, primers are designed to sandwich a polymorphic repeat sequence between the primers, and the primers are modified with fluorescence. Next, PCR is performed using these primers, the obtained product is electrophoresed on automatic fluorescence sequencer, and by measuring their chain lengths using a standard DNA as an indicator, polymorphism in the repeat sequence is detected. Recently, with the development of sequencers capable of processing a large number of samples, this method has become widely used, and is an effective means for detecting microsatellite polymorphisms.

Detection of polymorphism using mass spectrometry is also being performed. Until recently, mass spectrometry had been used mainly for testing the purity of synthetic oligonucleotides and not for detecting polymorphism due to reasons such as inability to quickly analyze a large number of samples and the assumed limit of the molecular weight of DNA that can be detected with this method. Testing the purity using mass spectrometry, a synthetic oligonucleotide specimen is directly applied with a matrix solution to a stainless steel platform or to a platform maintaining equivalent conductivity, dried, and then peak detection is carried out by matrix-assisted laser desorption ionization time-of-flight mass spectrometry (MALDI-TOF MS).

Detection of TNR polymorphism can also be performed using a mass spectrometer. According to this method, a genomic fragment to be detected is immobilized onto a platform by a silicone dioxide derivatization reaction, which is then subjected to the polymerase chain reaction using Primer-Oligo Based Extension Primers, followed by detection of the reactants using a mass spectrometer, and thereby determining the single nucleotide polymorphism (Kai Tang et al., Proc. Natl. Acad. Sci. USA Vol. 96, pp. 10016-10020, (1999)).

In case primers and/or probes are used for the amplification of PCR fragments, the primers and/or probes are chosen such that nucleotide sequence is complementary to a portion of a strand of an affected or a normal allele within about 150, preferably 100, most preferably 50, more preferably 20 nucleotides on either side of the GAG TNR, including directly adjacent to the GAG TNR region.

Preferably, the primer and/or probe for determining the presence of GAG TNR polymorphism will be selected from the group comprising any combinations of primers which amplify the GAG TNR polymorphic region in the GCLC gene.

Most preferably these primers and/or probes are 5′-TTCTGCGGGCGGCTGAGTGTCC-3′ (SEQ ID N^{o} 1) and 5′-ATGGCGCTTGGTTTCCTCCC-3′(SEQ ID N^{o} 2).

Also envisioned is the use of DNA chips or microarrays containing short DNA sequences immobilised at different positions. Such chips can be used to discriminate between alternative bases at the site of the GCLC GAG TNR. Briefly, a DNA sequence containing a single nucleotide polymorphism is hybridised to the chip. A method is employed to discriminate between alternative bases at the polymorphic site, usually based on the difference existing between the hybridization temperatures. A signal, corresponding to the specific identified GCLC GAG TNR, is detected. A chip can be used to type many polymorphisms simultaneously.

Two chip-based typing methods are widely used. One method relies on allele-specific hybridization. Short DNA sequences on the chip represent all possible variations at the GCLC GAG TNR polymorphic site; a labelled DNA will only stick if there is an exact match. The base is identified by the location of the fluorescent signal.

Alternatively, the oligonucleotide on the chip may stop one base before the variable site. In this case typing relies on allele-specific primer extension. A DNA sample stuck onto the chip is used as a template for DNA synthesis, with the immobilised oligonucleotide as a primer. The four nucleotides, containing different fluorescent labels, are added along with DNA polymerase. The incorporated base, which is inserted opposite to the polymorphic site on the template, is identified by the nature of its fluorescent signal. In a variation of this technique, the added nucleotide is identified not by a fluorescent label but by mass spectrometry.

Further encompassed in the present invention is a method for predicting susceptibility to a mental disorder, or a mental associated disorder, in a subject comprising i) obtaining a biological sample from said subject and, ii) determining the amino acid levels correlated with the presence of GAG TNR polymorphism in the 5′-untranslated region of the GCLC gene in said biological sample, iii) comparing said amino acid levels with those obtained from a control, iv) assessing whether the subject possesses a protective or a risk genotype associated with amino acid levels correlated with the presence of GCLC GAG TNR polymorphism, thereby determining whether the subject is susceptible to develop a mental disorder, or a mental associated disorder.

Usually, an increase in an amino acid level of at least one amino acid selected from alanine, aspartate, cystine (oxidized cysteine or Cys2), glutamate, glycine ornithine, phenylalanine, proline, serine, tyrosine, glutamine, arginine, citrulline, denotes a susceptibility to develop a mental disorder, or a mental associated disorder.

Preferably, a decrease in an amino acid level of at least one amino acid selected from reduced cysteine (Cysred), cysteine-glycine, hydroxyproline denotes a susceptibility to develop a mental disorder, or a mental associated disorder.

As described above, the biological sample is selected from the group comprising whole blood, semen, saliva, tears, urine, fecal material, sweat, buccal smears, skin, and biopsies of muscle, brain tissue, nerve tissue and hair. Preferably, the biological sample is cultured fibroblasts extracted from skin.

Additionally, the present invention also envisioned a kit for predicting susceptibility to a mental disorder, or a mental associated disorder, in a subject comprising
i) at least one primer and/or probe for determining the presence of GAG TNR polymorphism in the 5′-untranslated region of the GCLC gene in a biological sample, wherein said GAG TNR polymorphism is associated with a mental disorder, or a mental associated disorder,
ii) and optionally with reagents and/or instructions for use.

Preferably primers and/or probes used for the amplification of PCR fragments are chosen such that nucleotide sequence is complementary to a portion of a strand of an affected or a normal allele within about 150, preferably 100, most preferably 50, more preferably 20 nucleotides on either side of the GAG TNR, including directly adjacent to the GAG TNR region.

Preferably, the primer and/or probe for determining the presence of GCLC GAG TNR polymorphism will be selected form the group comprising any combinations of primers which amplify the GAG TNR polymorphic region in the GCLC gene. Most preferably the primer and/or probe is selected from the group comprising SEQ ID No 1 and 2.

The kit may further include at least one primer and/or probe for determining the presence of at least one polymorphism in a second gene involved in an epistatic interaction with GCLC. In such case, the primers and/or probes will be selected as described above adapted to the sequence of the second gene.

Preferably, the second gene involved in an epistatic interaction with GCLC is selected from the group of genes coding for Catechol O-methyltransferase, Dysbindin, Neuregulin-1, Protein kinase B, Disrupted in schizophrenia 1 protein, glutamic acid decarboxylase 1, Regulator of G-protein signalling 4, Receptor tyrosine-protein kinase erbB-4, Serine/threonine-protein phosphatase 2B catalytic subunit gamma isoform, Early growth response protein 3, Proline dehydrogenase 2, D-amino acid oxidase activator, Neuronal acetylcholine receptor subunit alpha-2, Glutamate-cysteine ligase modifiermodifier subunit, Glutathione synthetase, Glutathione peroxidase 1, Gamma-glutamyltranspeptidase 1, Nuclear respiratory factor 1, Nuclear factor erythroid 2-related factor or Nuclear factor erythroid 2-related factor 3.

Also encompassed in the present invention is a prognostic composition for predicting susceptibility to a mental disorder, or a mental associated disorder, in a subject comprising i) at least one primer and/or probe for determining the presence of GAG trinucleotide repeat (TNR) polymorphism in the 5′-untranslated region of the glutamate cystein ligase catalytic subunit (GCLC) gene in a biological sample, wherein said GAG trinucleotide repeat (TNR) polymorphism is associated with a mental disorder, or a mental associated disorder, ii) and optionally with reagents and/or instructions for use.

Preferably, the at least one primer and/or probe for determining the presence of GAG trinucleotide repeat (TNR) polymorphism will be selected as described above. More preferably, the primer and/or probe for determining the presence of GAG trinucleotide repeat (TNR) polymorphism will be selected from the group comprising SEQ ID No 1 or 2.

The prognostic composition of the invention will further comprise at least one primer and/or probe for determining the presence of at least one polymorphism in a second gene involved in an epistatic interaction with GCLC.

Preferably, the second gene involved in an epistatic interaction with GCLC is selected from the group of genes coding for Catechol O-methyltransferase, Dysbindin, Neuregulin-1, Protein kinase B, Disrupted in schizophrenia 1 protein, glutamic acid decarboxylase 1, Regulator of G-protein signaling 4, Receptor tyrosine-protein kinase erbB-4, Serine/threonine-protein phosphatase 2B catalytic subunit gamma isoform, Early growth response protein 3, Proline dehydrogenase 2, D-amino acid oxidase activator, Neuronal acetylcholine receptor subunit alpha-2, Glutamate-cysteine ligase modifier subunit, Glutathione synthetase, Glutathione peroxidase 1, Gamma-glutamyltranspeptidase 1, Nuclear respiratory factor 1, Nuclear factor erythroid 2-related factor or Nuclear factor erythroid 2-related factor 3.

Also with in the scope of the present invention is the use of the primer and/or probe for determining the presence of GAG trinucleotide repeat (TNR) polymorphism in the 5′-untranslated region of the glutamate cystein ligase catalytic subunit (GCLC) gene in the manufacture of a prognostic composition.

Further encompassed in the present invention is a kit for predicting susceptibility to a mental disorder, or a mental associated disorder, in a subject by determining the amino acid levels correlated with the presence of GAG TNR polymorphism in the 5′-untranslated region of the GCLC gene in said biological sample comprising means for the determination of the amino acid levels in a sample of a subject and means for comparing the said levels to reference levels.

Usually, means for means for the determination of the amino acid levels in a sample comprise solutions such as those described in example 3.

The invention further provides methods for treating, preventing, monitoring or delaying recurrence of a mental disorder, or a mental associated disorder. The methods comprise administering to a subject a pharmaceutical composition in a pharmaceutically effective amount for treating, preventing, monitoring or delaying said mental disorder, or said mental associated disorder.

The method of treatment of a mental disorder, or a mental associated disorder, comprises administering a pharmaceutical composition to a subject possessing a risk genotype associated with the presence of GAG trinucleotide repeat (TNR) polymorphism in the 5′-untranslated region of the glutamate cystein ligase catalytic subunit (GCLC) gene, thereby being susceptible to develop a mental disorder, or a mental associated disorder. Identifying critical period and specific brain location of GSH deficits, in particular with the method for predicting susceptibility of the invention, will help adjusting the dose and the administration manner of the pharmaceutical composition to said subject.

Preferably, the pharmaceutical composition comprises a pharmaceutically effective amount of at least one compound that increases GSH levels and/or activates GCL enzyme and selected from the group comprising:
Pro-drugs: H-Cys-Gly-OEt, H-Cys-Gly-OMe, H-Cys-Gly-OiPr, Ac-Cys-Gly-OH, Ac-Cys-Gly-OEt, H-γ-Glu-Cys-OEt, H-γ-Glu-Cys-OMet, H-γ-Glu-Cys-OiPr, Ac-γ-Glu-Cys-OH, Ac-γ-Glu-Cys-OEt, Ac-Cys-OH (N-Acetyl-Cysteine), Ac-Cys-OEt, Ac-Cys-ONH₂(N-Acetyl-Cysteine amide).
Pseudo-GSH, stabilized GSH: γ-Glu ψ [NHCO] Cys-Gly, γ-Glu-Cys ψ [NHCO] Gly, :γ-Glu-Cys-Gly-OEt (glutathione mono-ethyl ester, glutathione di-ethyl ester),
Pro-precursors: γ-Glu ψ [NHCO]-Cys, Cys ψ [NHCO]-Gly, γ-Glu-Cys-OEt
Non-peptidic precursor of Cys (or of Cys-Gly): R(−)-2-oxothiazolinine-4-carboxylic acid (OTC),
Substances known to increase GSH levels: Flupirtine, Vitamin E, Vitamine C, α-lipoic acid, bilirubin, curcumin and quercetin.
NADPH oxidase inhibitors: apocynin

The “pharmaceutically effective amount” refers to a chemical material or compound which, when administered to a subject induces a detectable pharmacological and/or physiologic effect.

The pharmaceutically effective amount of a dosage unit of the compound usually is in the range of 0.001 ng to 100 μg per kg of body weight of the patient to be treated.

Additionally, the pharmaceutical composition may contain one or more pharmaceutically acceptable carriers, diluents and adjuvants.

“Treatment”, as used herein, refers to both therapeutic treatment and prophylactic or preventative measures. Those in need of treatment include those already with the disorder as well as those in which the disorder is to be prevented. Hence, the subject to be treated herein may have been diagnosed as having the disorder or may be predisposed or susceptible to the disorder. Preferably, the subject is a human.

Various references are cited throughout this Specification, each of which is incorporated herein by reference in its entirety.

The foregoing description will be more fully understood with reference to the following Examples. Such Examples, are, however, exemplary of methods of practising the present invention and are not intended to limit the scope of the invention.

Examples

### Example 1

### Cell Culture

Penicillin/Streptomycin and Dulbecco's modified Eagle medium (DMEM), minimum was purchased from GIBCO (Live Technologies, Grand Island, N.Y.). Ultroser G was obtained from BioSepra (Biosystems, France). Sodium pyruvate was purchased from Sigma (Sigma-Aldrich, Switzerland). Western-Blots: Anti-human α-Tubulin, antibody was from Santa Cruz Biotechnology Inc. (Santa Cruz, Calif.). Antibodies for human GCLC and GCLM we obtained from Dr. Portia Vliet (University of Washington, Seattle). “Complete” protease inhibitor cocktail tablets were from Roche Diagnostics (Manheim, Germany). Protein concentration was determined by BCA protein assay (Pierce, Rockford, Ill.). PVDF Immobilion-P transfer membranes were purchased from Millipore. The enhanced chemoluminescence (ECL) detection system and Hyperfilms were purchased from Amersham International (Little Chalfont, UK). GSH content and GCL activity: 2,3-Naphthalenedicarboxyaldehyde was purchased from Fluka (Sigma-Aldrich, Switzerland). Glutathione, γ-glutamylcysteine, ATP, 5-sulfosalicylic acid, L-glutamic acid, L-cysteine, t-BHQ and other common reagents were purchased from Sigma (Sigma-Aldrich, Switzerland). GSH assay kit (Calbiochem, San Diego, Calif., USA).

### Subjects

All subjects of the Swiss cohort (66 patients and 48 controls, Table 1) were assessed using the Diagnostic Interview for Genetic Studies, developed by the NIMH [Nurnberger et al, 1994]. In the control group subjects with a history of a major psychiatric illness or having a first order relative with a history of psychosis were excluded. All subjects were of European Caucasian origin and matched for age. Sex was not matched, but all parameters were analyzed with sex as covariate for sex influences on variances (ANCOVA). No sex specific influence was observed. The recruited patients met the criteria for schizophrenia or schizoaffective disorder of the Diagnostic and Statistical Manual of Mental Disorders—Fourth Edition (DSM-IV). The subjects were recruited with fully informed written consent according to ethical guidelines of Lausanne University. The Danish cohort included 322 schizophrenia patients from the Danish Psychiatric Biobank, and 331 unrelated anonymous blood donors serving as healthy control subjects (Table 1). The recruited patients met the criteria for schizophrenia or schizoaffective disorder of the international Classification of Disorders (ICD-10).

### Cell Cultures and Treatment

Human fibroblast cultures were established from skin biopsies as described in Tosic et al.[8] Cells were grown with DMEM medium (0.5 l medium completed with 10 ml Ultroser-G serum, 5 ml Penicillin/Streptomycin, 5 ml sodium pyruvate (100 mM)) at 37° C. in a humidified atmosphere containing 5% CO2/95% air. Human fibroblasts near confluence after five passages were treated for 18 h with 50 μM t-BHQ in 0.05% DMSO (t-BHQ condition) or 0.05% DMSO alone (baseline condition).

### Western Blot Analysis

Cell lysis was performed on ice. Cells were washed with PBS and scraped with lysis buffer (2.37 ml scraping buffer (Tris-HCl 50 mM, pH 7.2; NaCl 150 mM; NaF 10 mM; EDTA 2 mM; EGTA 2 mM; Triton X100 1%); 100 μl Complete (1 tablet/2 ml H2O); 25 μl PMSF 100 mM; 2.5 μl DTT 1 mM). Lysates were homogenized 10 times with a 25-gauge needle, and cellular debris were cleared by centrifugation (20,000 g, 14 min, 4° C.). Protein concentration was determined by BCA protein assay. Equal amounts of protein (50 μg) were dissolved in 5× Laemmli sample buffer, separated by 12% SDS-PAGE, and electro-transferred to a PVDF membrane. Membranes were blocked (BSA 1%/milk powder 3%) overnight at 4° C. Following incubation with primary and secondary antibodies, protein was visualized by using the ECL detection system and protein abundance was quantified by densitometry analysis (Multi Genius, Bio Imaging System, Syngene). A fibroblast protein pool from 5 different individuals was used as an external control in all experiments. The results were normalized to a -Tubulin, and reported relative to the specific protein levels measured in the protein pool.

### GSH Content and GCL Activity

Cells were washed once with phosphate-buffer saline (PBS), trypsinized and pelleted. The pellet was washed once, taken up into PBS and frozen. GSH content and GCL activity was determined in protein extracts from fibroblasts cultures, with a fluorescence-based microtiter plate assay according to White and colleagues [White et al, 2003]. GCL activity was determined as the difference between GSH synthesis in unblocked and GSH synthesis in BSO treated wells per min and per mg protein. Protein concentration has been determined by BCA protein assay. In addition, GSH contents under untreated conditions were also routinely measured in all participants that had spent skin fibroblasts (33 controls and 39 patients) by a colorimetric GSH assay kit (Calbiochem, San Diego, USA).

### Genotyping (Method 1)

DNA was purified from blood samples using NucleonBACC3 system (Amersham Pharmacia Biotech). GCLC GAG TNR polymorphism was assessed by PCR, polyacrylamide gel electrophoresis and silver staining. PCR was performed with the primers GAG (−88 to −67) 5′-TTCTGCGGGCGGCTGAGTGTCC-3′ (SEQ ID N^{o} 1) and CTC (+35 to +54) 5′-ATGGCGCTTGGTTTCCTCCC-3′(SEQ ID N^{o} 2). PCR amplification was performed in reaction mixtures of 25 μl containing 40 ng of each primer, 250 μM of each NTP and 0.3 U EuroTaq DNA polymerase with its buffer (Euroclone, UK). Temperature cycling of the PCR was as follows: initial denaturation, 95° C. for 2 min; 5 cycles of 97° C. for 1 min, 65° C. for 1 mM, and 72° C. for 2 min; followed by 30 cycles of 95° C. for 1 min, 65° C. for 1 min, and 72° C. for 2 mM; and a final cycle of 7 mM at 72° C. PCR products were separated on an 8% polyacrylamide gel (6×8 cm) for 3 h with 120 V and gels were stained using SilverXpress Silver Staining Kit (Invitrogene, Carlsbad, USA).

### Genotyping (Method Walsh et al 1996; Radioactive Detection)

The primers used to amplify the region containing the trinucleotide repeat were 5′-TTCTGCGGGCGGCTGAGTGTCC-3′(SEQ ID N^{o} 1) (GAG strand) and 5′-ATGGCGCTTGGTTTCCTCCC-3′(SEQ ID N^{o} 2) (CTC strand) at position −88 to −67 and +35 to +54, respectively, relative to the start codon. PCR amplification was performed in reaction mixtures of 10 μl containing 50 ng of genomic DNA; 40 ng of each primer, 200 μM each of dATP, dGTP, and dTTP; 2.5 μM dCTP; 0.8 μCI [α-³²P] and 0.25 U AmpliTaq DNA polymerase in 1× PCR buffer (10 mM Tris-HCl [pH 8.3], 50 mM KCL, 1.5 mM MgCl₂, and 0.001% gelatin). Temperature cycling of the PCR was as follows: initial denaturation, 95° C. for 2 min; then 5 cycles of 97° C. for 1 min, 65° C. for 1 min, and 72° C. for 2 min; followed by 20 cycles of 95° C. for 1 min, 65° C. for 1 min, and 72° C. for 2 min; and a final cycle of 7 min at 72° C. Formamide gel loading dye was then added to the reaction mixture, the PCR products were denatured at 95° C. for 5 min, and then separated in a 6% polyacrylamide, 8.3 M urea sequencing gel. Allele sizes were determined by comparison with an M13 ladder. Based upon the GenBank sequence (accession No. L39773), the predicted size of the amplified product is 142 bp.

Genotying (Method Described in Bekris et al 2006: Detection with a 5-6-FAM-Labeled Primer; Radioactive Detection)

A 5-6-FAM-labeled primer was used along with a regular unlabeled primer. Using the labeled forward primer instead of a fluorescent R110 dCTP eliminated the need for a second polymerase chain reaction (PCR) step for incorporation and another purification step before microsatellite analysis. Fluorescence was incorporated into every PCR product with the labeled primer. The GC-RICH PCR System (Roche, Indianapolis, Ind.) was used for PCR amplification. PCR primers and probes sequences 5′-TTCTGCGGGCGGCTGAGTGTCC-3′ (SEQ ID N^{o} 1) and CTC (+35 to +54) 5′-ATGGCGCTTGGTTTCCTCCC-3′(SEQ ID N^{o} 2).

After 38 cycles of PCR amplification, the products were visualized on a 3% agarose gel stained with ethidium bromide. After ensuring that the products are present and there is no contamination in any negative controls, the products were further analyzed on a 5% polyacrylamide gel using an ABI PRISM 377 DNA Sequencer (PE Applied Biosystems). Samples were then scanned using GeneScan 3.1 (PE/ABI) and genotypes were analyzed using Genotyper 2.0 (PE/ABI).

### Statistical Analysis

Statistical analysis has been performed using the SAS statistical package version 9 (SAS Institute Inc., Cary, N.C., USA). The distribution of the protein levels were tested for normality (Univariate procedure, SAS Users Guide: Base, 1989) and data were transformed with an exponential function in order to obtain normally distributed values. A two way ANOVA was used to test the effect of the group and treatment (GLM procedure, SAS/STAT User's Guide, version 6, 1989). The treatment effect was studied also with a MANOVA model in which the baseline values were taken as covariables. The correlations between the protein levels were studied using the CORR procedure and correction for multiple testing was applied when necessary. Genotype distribution between patients and controls was tested using Chi-Square test (two-tailed), or if cells had expected count less than 5, the Fisher exact test (two-tailed). Mann-Whitney U test (two-tailed) was used to compare the means of the groups of the TNR polymorphism for GCL activity, GCLC protein expression and GSH content. Boxplot graphs were produced using the SPSS statistical package, version 14.0 (SPSS Inc., Chicago, Ill., USA).

### Example 2

### Results

### Tables

TABLE 1
Demographic characteristics of the groups of
patients and controls used in the study

| | | | | |
|---|---|---|---|---|
| Cohort, | | | Sex | |
| Samples & Study | N | Age | (m/f) | Diagnostic |

A) Swiss cohort; Skin fibroblasts ± t-BHQ treatment
([GSH], GCL activity, protein)

| | | | | |
|---|---|---|---|---|
| Controls | 26 | 36.6 ± 12.4 | 1.2 | DIGS |
| Patients | 26 | 36.7 ± 11.1 | 3.3 | DSM-IV, DIGS |

B) Swiss cohort, Blood samples: (genotyping)

| | | | | |
|---|---|---|---|---|
| Controls | 48 | 34.3 ± 11.6 | 1.2 | DIGS |
| Patients | 66 | 33.9 ± 10.7 | 2.8 | DSM-IV, DIGS |

C) Swiss cohort; Skin fibroblasts: (baseline [GSH])

| | | | | |
|---|---|---|---|---|
| Controls | 39 | 35.7 ± 10.4 | 1.1 | DIGS |
| Patients | 33 | 35.3 ± 13.2 | 2.5 | DSM-IV, DIGS |

D) Danish cohort; Blood samples: (genotyping)

| | | | | |
|---|---|---|---|---|
| Controls | 331 | 40.2 ± 10.5 | 1.5 | Random selection |
| Patients | 322 | 38.8 ± 12.1 | 1.5 | ICD-10 |

Group A and C are both part of group B.
Age is presented as mean ± standard deviation.
Sex as the ratio between male and female.
DSM-IV = Diagnostic system manual IV.
DIGS = Diagnostic Interview for Genetic Studies

TABLE 2
Comparison of GCL activity, GSH contents, GCLM, and GCLC protein
expression in schizophrenia patients vs. control subjects

| | | Untreated | | t-BHQ | |
|---|---|---|---|---|---|
| Variable | N | Controls | Patients | Controls | Patients |
| GCL activity | 25 | 0.250 ± 0.08 | 0.215 ± 0.06 | 0.847 ± 0.26 | 0.627 ± 0.16** |
| GSH content | 25 | 56.7 ± 8.0 | 53.6 ± 6.4 | 144.9 ± 21.5 | 136.3 ± 19.1 |
| GCLM | 26 | 62.3 ± 16.3 | 61.7 ± 11.3 | 108.0 ± 24.0 | 107.6 ± 24.1 |
| GCLC | 26 | 44.4 ± 12.3 | 34.5 ± 10.9** | 64.1 ± 17.5 | 45.4 ± 15.8*** |

N is the number of tested subjects.
Values are given as average and standard deviation under untreated or t-BHQ treated conditions.
GSH contents are shown as nmol GSH/mg protein.
GCL activity is expressed as nmol GSH/min/mg protein.
GCLM and GCLC protein expressions are presented as α-Tubulin corrected arbitrary units.
GCL activity under t-BHQ treated and GCLC protein expression under both untreated, as well as t-BHQ treated conditions were significantly decreased in patients, as compared to controls.
**P < 0.01
***P < 0.001 versus the respective controls were calculated using ANOVA test (two-tailed).

TABLE 3
Distribution of GCLC GAG TNR genotypes in Swiss cohort
of schizophrenia patients and control subjects

| | Controls | | Patients | | | | |
|---|---|---|---|---|---|---|---|
| Genotypes | N | % | N | % | Odds Ratio | 95% CI | P Value |
| 7/7 | 24 | 50.0 | 20 | 30.3 | 0.44 | 0.20-0.94 | 0.033 |
| 7/9 | 21 | 43.7 | 22 | 33.3 | 0.64 | 0.30-1.38 | 0.257 |
| 7/8 | 2 | 4.2 | 13 | 19.7 | 5.64 | 1.21-26.32 | 0.015 |
| (8/8, 8/9, | 1 | 2.1 | 11 | 16.7 | 9.40 | 1.17-75.53 | 0.012 |
| 9/9) | | | | | | | |

| | |
|---|---|
| *Controls vs. Patients: (24, 21, 2, 1 vs. 20, 22, 13, 11) | 0.002 |

N is the number, and % the percentage of genotypes in a group. Rare genotypes 8/8, 8/9 & 9/9 were grouped together. Data are given together with odds ratio and 95% confidence interval (CI).
P values were calculated using Chi-Square test (two-tailed), or if cells had expected count less than 5, the Fisher exact test (two-tailed).
*Frequency of the genotypes between patients and controls was compared using Fisher exact test (two-tailed) with a 2 × 4 contingency table (Controls & Patients × 7/7 & 7/8 & 7/9 & (8/8, 8/9, 9/9) genotypes).

TABLE 4
Distribution of GCLC GAG TNR genotypes in Danish
cohort of schizophrenia patients and control subjects

| | Controls | | Patients | | | | P |
|---|---|---|---|---|---|---|---|
| Genotypes | N | % | N | % | Odds Ratio | 95% CI | Value |
| 7/7 | 125 | 37.8 | 87 | 27.0 | 0.61 | 0.43-0.85 | 0.003 |
| 7/8 | 65 | 19.6 | 64 | 19.9 | 1.02 | 0.69-1.49 | 0.939 |
| 7/9 | 86 | 26.0 | 103 | 32.0 | 1.34 | 0.95-1.88 | 0.091 |
| 8/8 | 8 | 2.4 | 22 | 6.8 | 2.96 | 1.30-6.75 | 0.007 |
| 8/9 | 27 | 8.2 | 19 | 5.9 | 0.71 | 0.38-1.30 | 0.260 |
| 9/9 | 20 | 6.0 | 27 | 8.4 | 1.42 | 0.78-2.59 | 0.247 |

| | |
|---|---|
| *Controls vs. Patients: (125, 65, 86, 8, 27, | 0.004 |
| & 20 vs. 87, 64, 103, 22, 19 & 27) | |

N is the number, and % the percentage of genotypes in a group.
Data are given together with odds ratio and 95% confidence interval (CI).
P values were calculated using Chi-Square test (two-tailed).
*Frequency of the genotypes between patients and controls was compared using Chi-Square test (two-tailed) with a 2 × 6 contingency table (Controls & Patients × 7/7, 7/8, 7/9, 8/8, 8/9 & 9/9 genotypes).

### GCL Activity and GSH Content

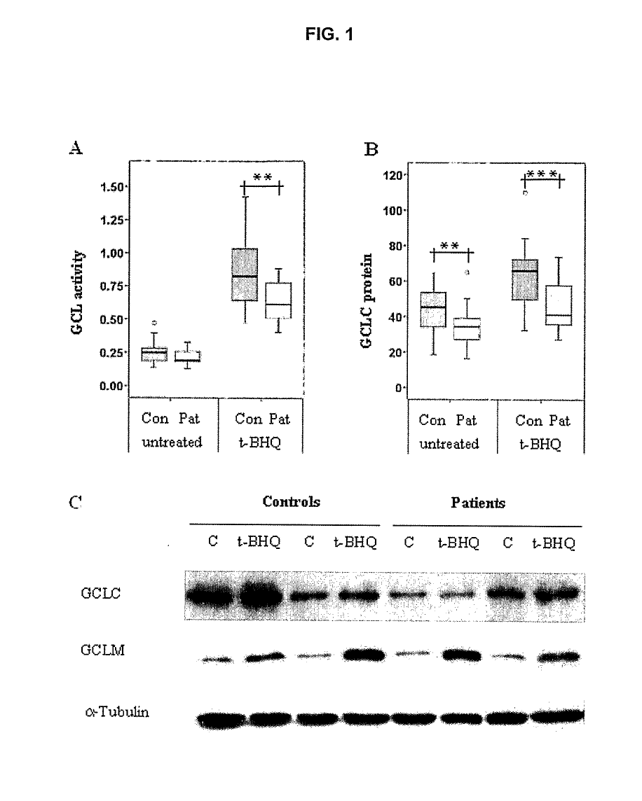

GCL activity and GSH content were measured under baseline and t-BHQ treated conditions in cultured skin fibroblasts derived from schizophrenia patients and control subjects (FIG. 1A, Table 2). Under t-BHQ treated conditions GCL activity was decreased in patients by 26% (P=0.002), as compared to controls, while under baseline conditions there was a trend toward a decrease. After t-BHQ treatment GCL activity in controls increased by a factor of 3.45, while it increased in patients by a factor of 2.95 only. Thus patients displayed a significant (P=0.001) deficit in their ability to react against oxidative stress. The increase of GSH content by a factor of 2.53 after t-BHQ treatment was very similar in patients and controls. GSH levels under baseline and t-BHQ treated conditions tended to be lower in patients than in controls, but the difference was not significant.

### GCLC and GCLM Protein Expression

In parallel to measurements of GCL activity and GSH content, protein levels of GCLM and GCLC were quantified by Western Blot analysis (FIGS. 1B & C, Table 2). GCLC protein expression in patients, as compared to controls, was decreased under baseline conditions by 22% (P=0.009) and after t-BHQ treatment by 29% (P<0.001). T-BHQ treatment increased GCLC protein expression in controls by a factor of 1.44, while the increase in patients was significantly less (P=0.005), by a factor of only 1.32. In contrast, GCLM protein expression did not differ between patients and controls, and the increase induced by t-BHQ treatment was the same: 73% in control subjects and 74% in patients.

### Regulation of GSH Synthesis in Patients and Controls: Statistical Analysis

GSH content, GCL activity, and GCLC and GCLM protein expression are all parameters of GSH synthesis. With the aim to understand interactions between individual parameters of the GSH synthesis in response to t-BHQ treatment and in relation to patients and controls, we performed statistical analysis. In a first step we hypothesized that the ratio of GCLM/GCLC protein levels corresponds with the functional GCL holoenzyme. We tested the influence of the group (patient vs. control), the treatment (with or without t-BHQ) and the ratio of GCLM/GCLC protein expression on the two variables “GSH content” and “GCL activity” by a two-way ANOVA model. Results showed that GSH content and GCL activity were significantly influenced by the group (P=0.018), by the ratio of GCLM/GCLC protein expression (P=0.021), and by the treatment (P<0.001). This statistical analysis confirmed a significant interaction between the treatment and the group (P=0.029). This interaction can be do to different relationships between GCLC protein level and GCL activity inside the groups of patients and controls.

In a second step we performed multivariate analysis of variances (MANOVA) for the following dependent variables: “GCLM protein expression”, “GCLC protein expression”, “GCL activity”, and “GSH content”. Results confirmed that under conditions of t-BHQ treatment GSH synthesis was significantly (P=0.019) different between patients and controls. Univariate analysis of the same parameters (ANOVA) revealed the following effects: a) GCL activity after t-BHQ treatment was influenced significantly (P=0.025) by the degree of GCLC protein expression present before the treatment; b) GCL activity was differently regulated (P<0.001) in patients vs. controls; c) in both patients and controls, the GSH content after t-BHQ treatment was influenced by the baseline protein expression of GCLC (P=0.032); d) the level of GCLC protein expression after t-BHQ treatment was influenced (P=0.011) by the level of GCLM protein expression before t-BHQ treatment; and e) GCLC protein expression was significantly different (P<0.001) between patients and controls.

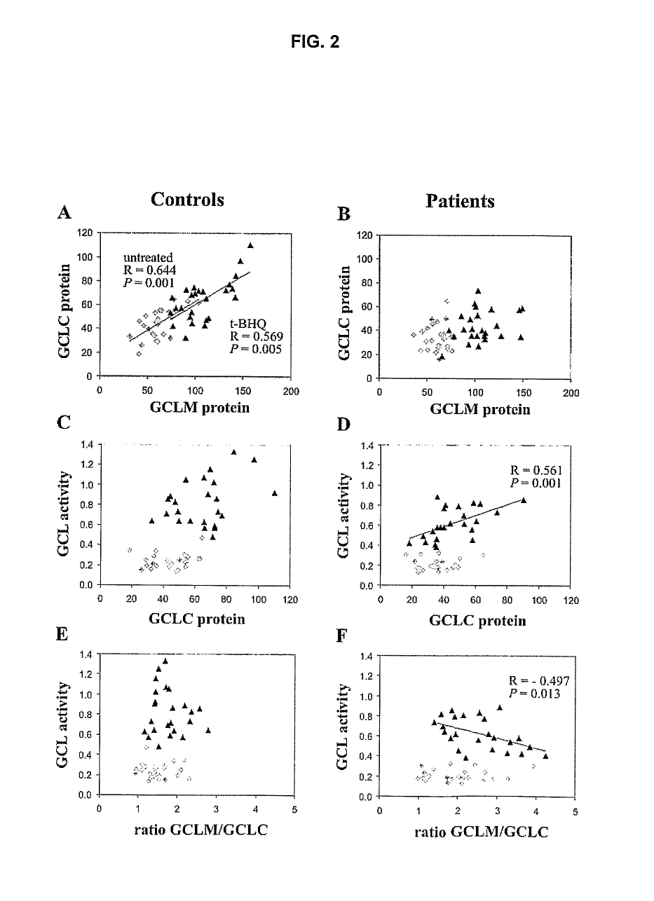

Aside from the impaired GCLC protein expression in patients, we consistently observed a different regulation of GCLM and GCLC protein expression in patients as compared to controls: In control subjects protein expression of GCLM and GCLC was correlated under untreated as well as under t-BHQ treated conditions (FIG. 2A). These correlations, however, were lost in patients (FIG. 2B). The impaired GCLC protein expression in patients under t-BHQ treated conditions correlated with GCL activity (FIG. 2D) and was thus limiting for GSH synthesis and for GSH content (R=0.435; P=0.034). These correlations were not observed in controls (FIG. 2C). Finally, analysis of correlations between the ratio of GCLM/GCLC with GCL activity or with GSH content under t-BHQ treated conditions revealed a different regulation between patients and controls. In patients, both GCL activity (FIG. 2F) and GSH content (R=−0.481; P=0.017) were inversely correlated with the GCLM/GCLC ratio, while this correlation was not observed in controls (FIG. 2E).

### GAG TNR Polymorphism in GCLC Gene; Swiss Cohort

The GCLC gene was reported to contain a GAG TNR polymorphism just 10 base pairs upstream of the start codon. We investigated this polymorphism in the Swiss cohort (66 schizophrenia patients and 48 control subjects) and we compared the frequency of the genotypes between patients and controls (Table 3). It should be noted that control subjects in this cohort have been selected according to the DIGS, excluding subjects with a history of a major psychiatric illness or having a first order relative with a history of psychosis. In comparison to genotypes 7/7, 7/8 & 7/9, genotypes 8/8, 8/9, & 9/9 were rare (six 8/9; four 9/9 and two 8/8). Results revealed that 50.0% of the control subjects had a 7/7 genotype, 43.7% a 7/9, 4.2% a 7/8, and 2.1% an 8/9 genotype. 45 out of 48 control subjects had no 8 TNR allele and genotypes 8/8 and 9/9 were absent. In contrast, 24 of 66 (36%) patients had a genotype with an 8 TNR allele, or had genotype 9/9, resulting in a significant (P=0.002) different genotype distribution in patients (30.3% 7/7, 33.3% 7/9, 19.7% 7/8, 7.6% 8/9, 3.0% 8/8, & 6.1% 9/9). In particular, as compared to controls, patients had significantly less often (P=0.033) a genotype 7/7, but had significantly more often (P=0.015) a genotype 7/8, 8/8, 8/9, or 9/9 (P=0.012).

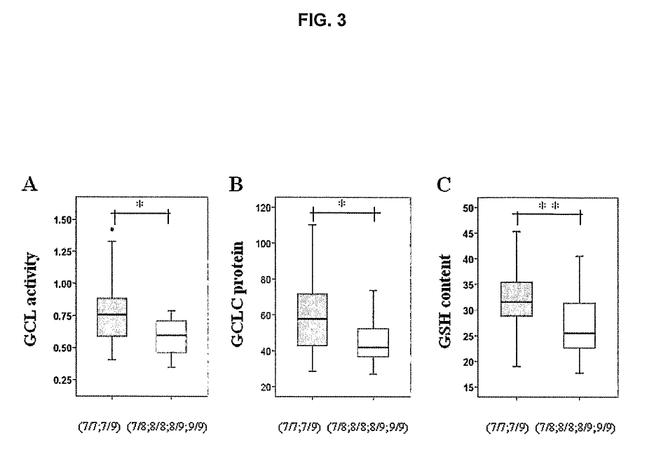

As genotypes 7/7 and 7/9 were more frequent in control subjects, while all other genotypes were present more often in patients, we regrouped the data of GCL activity and GCLC protein expression (both under conditions of t-BHQ treatment) of all 52 subjects according their genotypes (7/7 & 7/9 vs. 7/8, 8/8, 8/9, & 9/9) and we compared the parameters between these two groups (FIGS. 3A&B). The group of genotypes 7/7 and 7/9 had a higher GCL activity (0.784±0.246 vs. 0.584±0.141; P=0.017), as well as a higher GCLC protein expression (58.9±19.0 vs. 45.6±13.3; P=0.037) as compared to the group of genotypes 7/8, 8/8, 8/9, & 9/9.

As we have measured fibroblast GSH contents of all participants for which skin fibroblasts were obtained, we were able to analyze the effect of the GCLC GAG TNR genotypes on the baseline GSH contents of 72 subjects (33 controls and 39 patients, Table 3). The group of 7/7 and 7/9 genotypes had a significant higher baseline GSH content (32.1±5.8 vs. 27.0±6.0; P=0.004), as compared to the group of all other genotypes (FIG. 3C).

### GAG TNR Polymorphism in GCLC Gene; Danish Cohort

In an additional case-control study we tested the distribution of the GAG genotypes in a Danish cohort of 322 schizophrenia patients and 331 unrelated anonymous blood donors serving as healthy control subjects. Results also showed a significantly (P=0.004) different genotype distribution between patients and controls (Table 4). Patients of the Danish cohort had a very similar genotype distribution, as compared to patients of the Swiss cohort. In contrast, the randomly chosen control subjects of the Danish population had a different genotype distribution, as compared to controls of the Swiss population, where subjects with a history of a major psychiatric illness or with a first order relative that had a psychosis were excluded. In particular, the results showed that genotype 8/8 was significantly (P=0.007) associated with schizophrenia. On the other side genotype 7/7 again was present significantly (P=0.003) more often in controls than in patients.

### Example 3

### Amino Acid Dosage

Blood preparation

Blood is collected by venipuncture between 7 and 8:30 AM under restricted activity conditions and fasting from the previous midnight. 18-20 ml blood is allowed to drop in Vacutainer-tubes coated with EDTA (Becton Dickinson) which are previously placed on ice. Hemoglobin is quantified. All the following preparation must be done on ice or at 4° C. An aliquot of whole blood is sampled and freezed at −80° C. until analysis of GSH content. The rest of blood is centrifuged at 3000 g, 5 min, 4° C.; the pellet, corresponding to blood cells, is washed 2 times with 0.9% NaCl and freezed at −80° C. until analysis. The supernatant, corresponding to the plasma, is recovered, sampled in aliquots and kept at −80° C. until analysis.

### Fibroblast Preparation for Biochemical Assays

Cultures of fibroblast (4 Petri dishes of 10 cm diameter, confluent) established from skin biopsies as described in Tosic et al, 2006, were collected after 3 passages. They were removed from flasks with trypsin, washed, resuspended in 4 ml phosphate buffer (0.1M, pH7.4) and sonicated. Aliquots from this homogenate were kept at −80° C. for GSH and protein determination. The rest was centrifuged at 5000 g for 10 min at 4° C. The supernatant was sampled in 100 μl aliquots which were used for GSH-related enzymes activity determination.

### Protein Determination

The protein levels of fibroblasts were determined using the Biorad Kit with the Advanced Protein Assay reagent in accordance with the manufacturer's instructions.

### Glutathione Determination

The glutathione (GSH) levels in blood cells, plasma or fibroblasts was determined using a diagnostic kit purchased from Calbiochem. This method is based on a colorimetric assay of a chromophoric thione formed specifically between the reagent and GSH.

### Amino Acid Analysis in Plasma

The following free amino acids were quantified in plasma as described in Slocum et al., 1991: Taurine (Tau), Aspartic acid (Asp), Hydroxyproline (Hyp), Threonine (Thr), Serine (Ser), Asparagine (Asn), Glutamic acid (Glu), Glutamine (Gln), Proline (Pro), glycine (Gly), Alanine (Ala), Citrulline (Cit), Aminobutyric acid (Abu), Valine (Val), Cystine (Cyt), Methionine (Met), Isoleucine (Ile), Leucine (Leu), Tyrosine (Tyr), Phenylalanine (Phe), Ornithine (orn), Lysine (Lys), 1-CH₃-Histidine (1-CH₃-His), Histidine (His), 3-CH₃-Histidine (3-CH₃-His), Arginine (Arg)

In brief, the plasma containing (S)-2-Aminoethyl-1-cysteine and d-glucosaminic acid (1.25 mM) as internal standards was deproteinated by 5-sulfosalicylic acid and kept at −80° C. until analysis. 200 μl of plasma are put in a 0.6 ml centrifuge tube, then 20 μl SSA solution (35% w/v) are added. The tube is vortexed for 5 sec, and after 5 min at room temperature it is centrifuged at 11′200 g for 5 min. the supernatant is drawn off above the protein precipitate. Then the sample is Centrifuged at 1800 g for 30 min in a capped Centrifree (Amicon, Danvers, Mass.) microfilter reservoir. 200 μl D-glucosaminic acid (0.1 mg in 2.0 ml of pH 2.2 Beckman Li—S buffer (Beckman 338084, Beckman Instruments, Inc., Palo Alto, Calif.) or equivalent are added to a 200 μl aliquot of the sample. This serves as a quality control standard for the amount of sample injected into the amino acid analyzer. It is then injected to an amino acid analyser (Beckman, 6300 Model; column Lithium, 10 cm, Beckman 338051) and detected by post-column reaction with nihydrine.

### Thiols Analysis in Plasma

The following thiol containing amino acids and peptide were quantified in plasma as described in Vester B, Rasmussen K.1991: Cysteine (total: reduced+oxidised form), Homocysteine and Cysteinyl-glycine

In brief, the thiols were reduced and/or decoupled from proteins by reaction with Tris(2-carboxyethyl)phosphine. After deproteinization with perchloric acid, the thiols were derivatised with 7-fluorobenzofurazane-4-sulfonic acid (SBD-F). The SBD-F derivatives were analysed by HPLC followed by fluorometric detection.

### Results

Amino acid levels discriminate patients from controls. Plasma levels of 24 amino acids have been assessed in 67 healthy control subjects and in 56 schizophrenia patients (Table 5). Comparing amino acid levels of patients and controls with a logistic regression revealed a clear discrimination with a sensitivity of 93.5% and a selectivity of 6.4%. MANOVA revealed that amino acid levels were influenced by the disease, age and sex (P<0.0001). Independent of the influences of age and sex patients had different amino acid levels (P<0.0001), as compared to controls. ANOVA, controlled for age and sex, confirmed for patients significant increased amino acid levels of alanine, aspartate, cystine, glutamate, glycine, ornithine, phenylalanine, proline, serine, and tyrosine. Cysteine-glycine and hydroxyproline levels were significantly lower in patients than in controls (Table 5).

TABLE 5
Serum amino aid levels in patients and controls

| Amino acids | Control subjects | Schizophrenia patients | ANOVA (P) |
|---|---|---|---|
| Alanine | 329.0 ± 76.4 | 371.1 ± 100.7 | 0.015 |
| Arginine | 76.3 ± 14.6 | 82.7 ± 22.1 | n.s. |
| Asparagine | 46.4 ± 7.8 | 47.8 ± 13.4 | n.s. |
| Aspartate | 3.6 ± 1.0 | 4.9 ± 1.7 | <0.001 |
| Citrulline | 29.1 ± 7.4 | 29.9 ± 8.6 | n.s. |
| Cystine | 36.3 ± 16.2 | 43.8 ± 22.0 | 0.043 |
| Cysteine- | 45.9 ± 8.6 | 43.3 ± 8.0 | 0.041 |
| Glycine | | | |
| Cysteine | 188.8 ± 44.1 | 179.2 ± 49.8 | n.s. |
| Glutamine | 512.1 ± 84.4 | 556.8 ± 127.6 | n.s. |
| Glutamate | 31.7 ± 17.6 | 57.8 ± 43.5 | <0.001 |
| Glycine | 198.9 ± 49.2 | 233.9 ± 74.2 | 0.003 |
| Homocysteine | 10.9 ± 11.5 | 9.3 ± 2.6 | n.s. |
| Histidine | 75.4 ± 12.4 | 80.3 ± 19.2 | n.s. |
| Hydroxyproline | 15.7 ± 10.4 | 9.8 ± 5.3 | <0.001 |
| Isoleucine | 61.6 ± 15.9 | 65.8 ± 20.2 | n.s. |
| Leucine | 121.2 ± 25.0 | 128.7 ± 36.2 | n.s |
| Lysine | 164.3 ± 26.2 | 172.9 ± 48.4 | n.s |
| Methionine | 22.6 ± 4.4 | 23.4 ± 5.9 | n.s |
| Ornithine | 44.0 ± 11.8 | 55.0 ± 20.8 | <0.001 |
| Phenylalanine | 44.1 ± 18.9 | 52.5 ± 15.9 | 0.008 |
| Proline | 175.0 ± 55.3 | 212.6 ± 62.7 | 0.005 |
| Serine | 89.0 ± 19.0 | 98.1 ± 26.7 | 0.047 |
| Taurine | 51.0 ± 17.4 | 50.6 ± 15.9 | n.s |
| Threonine | 123.3 ± 29.7 | 125.9 ± 48.5 | n.s |
| Tyrosine | 56.0 ± 12.0 | 62.6 ± 17.7 | 0.036 |
| Valine | 217.8 ± 41.3 | 238.3 ± 63.4 | n.s |

Serum amino acid levels were assessed in 67 control subjects and 56 schizophrenia patients and are presented in μmol/l as means ± StDev.
Multivariate analysis of variance (MANOVA) procedure was assessed using the amino acid levels as dependent variables and sex, age and status as independent variables.
P were tested as univariate analysis of variance (ANOVA) as part of the MANOVA procedure.
n.s. not significant.

### Example 4

### GAG TNR and Associated Mental Disorders

### Lausanne Cohort

Applicants have genotyped the GAG TNR polymorphism of the GCLC gene in 71 controls, 74 schizophrenia, 62 major depression, and 107 bipolar disorder patients.

In line with the results shown throughout the description, the GAG TNR polymorphism is distributed differently between schizophrenia patients and controls in the Lausanne sample, as well as in the Danish sample (726 controls, 488 patients, P=0.0004).

Apart of schizophrenia, also bipolar disorder patients have a different GAG genotype distribution in comparison to controls.

Comparing the GCLC GAG TNR genotype distribution between healthy controls and other groups of psychiatric patients revealed an association with bipolar disorder (P=0.010), but not with major depression (P=0.381).

TABLE 6
Distribution of GCLC GAG TNR genotypes in
Lausanne cohort of schizophrenia patients,
bipolar disorders patients and control subjects

| | | Schizophrenia | Bipolar disorder |
|---|---|---|---|
| Genotypes | Controls | Patients | patients |
| 7/7 | 36 | 25 | 40 |
| 7/9 | 28 | 24 | 35 |
| 7/8 | 2 | 14 | 21 |
| 8/9 | 2 | 5 | 7 |
| 8/8 | 0 | 2 | 2 |
| 9/9 | 3 | 4 | 2 |
| Total | 71 | 74 | 107 |
| Chi-Square | | 0.012 | 0.01 |
| (GAG) | | | |

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

### REFERENCES

Owen M J, Craddock N, O'Donovan M C (2005) Schizophrenia: genes at last?, Trends Genet 21:518-525
Do K Q, Lauer C J, Schreiber W, Zollinger M, Gutteck-Amsler U, Cuenod M, Holsboer F (1995) gamma-Glutamylglutamine and taurine concentrations are decreased in the cerebrospinal fluid of drug-naive patients with schizophrenic disorders, J Neurochem 65:2652-2662
Do K Q, Trabesinger A H, Kirsten-Kruger M, Lauer C J, Dydak U, Hell D, Holsboer F, Boesiger P, Cuenod M (2000) Schizophrenia: glutathione deficit in cerebrospinal fluid and prefrontal cortex in vivo, Eur J Neurosci 12:3721-3728
Yao J K, Leonard S, Reddy R (2006) Altered glutathione redox state in schizophrenia, Dis Markers 22:83-93
Tosic M, Ott J, Barral S, Bovet P (2006) Schizophrenia and oxidative stress: Glutamate cysteine ligase modifier as a susceptibility gene, Am J Hum Gen 79:586-592
Soltaninassab S R, Sekhar K R, Meredith M J, Freeman M L (2000) Multi-faceted regulation of gamma-glutamylcysteine synthetase, J Cell Physiol 182:163-170
Satoh T, Okamoto S I, Cui J, Watanabe Y, Furuta K, Suzuki M, Tohyama K, Lipton S A (2006) Activation of the Keap1/Nrf2 pathway for neuroprotection by electrophilic [correction of electrophillic] phase II inducers, Proc Natl Acad Sci USA 103:768-773
Meister A (1995) Glutathione biosynthesis and its inhibition, Methods Enzymol 252:26-30
Walsh A C, Feulner J A, Reilly A (2001) Evidence for functionally significant polymorphism of human glutamate cysteine ligase catalytic subunit: association with glutathione levels and drug resistance in the National Cancer Institute tumor cell line panel, Toxicol Sci 61:218-223
Nurnberger J I, Jr., Blehar M C, Kaufmann C A, York-Cooler C, Simpson S G, Harkavy-Friedman J, Severe J B, Malaspina D, Reich T (1994) Diagnostic interview for genetic studies. Rationale, unique features, and training. NIMH Genetics Initiative, Arch Gen Psychiatry 51:849-859
White C C, Viernes H, Krejsa C M, Botta D, Kavanagh T J (2003) Fluorescence-based microtiter plate assay for glutamate-cysteine ligase activity, Anal Biochem 318:175-180
Walsh A C, Li W, Rosen D R, Lawrence D A (1996) Genetic mapping of GLCLC, the human gene encoding the catalytic subunit of gamma-glutamyl-cysteine synthetase, to chromosome band 6p12 and characterization of a polymorphic trinucleotide repeat within its 5′ untranslated region, Cytogenet Cell Genet 75:14-16
Sokoloff L (1999) Energetics of functional activation in neural tissues, Neurochem Res 24:321-329
Robertson G S, Hori S E, Powell K J (2006) Schizophrenia: an integrative approach to modelling a complex disorder, J Psychiatry Neurosci 31:157-167
Piazza P V, Le Moal M L (1996) Pathophysiological basis of vulnerability to drug abuse: role of an interaction between stress, glucocorticoids, and dopaminergic neurons, Annu Rev Pharmacol Toxicol 36:359-378
Mimics K, Levitt P, Lewis D A (2006) Critical appraisal of DNA microarrays in psychiatric genomics, Biol Psychiatry 60:163-176
Yang Y, Dieter M Z, Chen Y, Shertzer H G, Nebert D W, Dalton T P (2002) Initial characterization of the glutamate-cysteine ligase modifier subunit Gclm(−/−) knockout mouse. Novel model system for a severely compromised oxidative stress response, J Biol Chem 277:49446-49452
Koide S, Kugiyama K, Sugiyama S, Nakamura S, Fukushima H, Honda O, Yoshimura M, Ogawa H (2003) Association of polymorphism in glutamate-cysteine ligase catalytic subunit gene with coronary vasomotor dysfunction and myocardial infarction, J Am Coll Cardiol 41:539-545
Beutler E, Gelbart T, Kondo T, Matsunaga A T (1999) The molecular basis of a case of gamma-glutamylcysteine synthetase deficiency, Blood 94:2890-2894
Dahl N, Pigg M, Ristoff E, Gali R, Carlsson B, Mannervik B, Larsson A, Board P (1997) Missense mutations in the human glutathione synthetase gene result in severe metabolic acidosis, 5-oxoprolinuria, hemolytic anemia and neurological dysfunction, Hum Mol Genet 6:1147-1152
Dalton T P, Dieter M Z, Yang Y, Shertzer H G, Nebert D W (2000) Knockout of the mouse glutamate cysteine ligase catalytic subunit (Gclc) gene: embryonic lethal when homozygous, and proposed model for moderate glutathione deficiency when heterozygous, Biochem Biophys Res Commun 279:324-329
Nakamura S, Kugiyama K, Sugiyama S, Miyamoto S, Koide S, Fukushima H, Honda O, Yoshimura M, Ogawa H (2002) Polymorphism in the 5′-flanking region of human glutamate-cysteine ligase modifier subunit gene is associated with myocardial infarction, Circulation 105:2968-2973
Curkendall S M, Mo J, Glasser D B, Rose S M, Jones J K (2004) Cardiovascular disease in patients with schizophrenia in Saskatchewan, Canada, J Clin Psychiatry 65:715-720
Culverhouse R, Klein T, Shannon W (2004) Detecting epistatic interactions contributing to quantitative traits, Genetic Epidemiology 27:141-152
Slocum R H, Cummings J G: Amino Acid Analysis of Physiological samples. In “Tech-niques in diagnostic human Biochemical Genetics. Hommes Edt. 1991, pp 87-126
Bekris L M, Viernes H M, Farin F M, Maier L A, Kavanagh T J, Takaro T K. (2006) “Chronic beryllium disease and glutathione biosynthesis genes” in J Occup Environ Med. 2006 June; 48(6):599-606
Vester B, Rasmussen K. High performance liquid chromatography method for rapid and accurate determination of homocysteine in plasma and serum. in Eur J Clin Chem Clin Biochem. 1991 September; 29(9):549-54

1. A method for predicting susceptibility to a mental disorder, or a mental associated disorder, in a subject comprising
i) obtaining a biological sample from said subject and,
ii) determining at least the presence of GAG trinucleotide repeat (TNR) polymorphism in the 5′-untranslated region of the glutamate cystein ligase catalytic subunit (GCLC) gene in said biological sample,
iii) assessing whether the subject possesses a protective or a risk genotype associated with the presence of GAG trinucleotide repeat (TNR) polymorphism in the 5′-untranslated region of the glutamate cystein ligase catalytic subunit (GCLC) gene, thereby determining whether the subject is susceptible to develop a mental disorder, or a mental associated disorder.

2. The method of claim 1 wherein step ii) further comprises the determination of at least one polymorphism in a second gene involved in an epistatic interaction with GCLC.

3. The method of claim 1 wherein the GAG trinucleotide repeat (TNR) polymorphism in the 5′-untranslated region of the glutamate cysteine ligase catalytic subunit (GCLC) gene consists in 7, 8 or 9 GAG repeats.

4. The method of claim 1 wherein the GAG trinucleotide repeat (TNR) polymorphism in the 5′-untranslated region of the glutamate cystein ligase catalytic subunit (GCLC) gene in said biological sample is associated with lower glutathione GSH content, and/or decreased level of GCLC protein and/or a lower capacity to synthesize GSH protein and/or modified amino acid levels.

5. The method of claim 1, wherein the mental disorder is selected from the group of schizophrenic disorders, affective disorders, psychoactive substance use disorders, personality disorders, delirium, dementia, epilepsy, panic disorder, obsessive compulsive disorder, intermittent explosive disorder, impulse control disorder, psychosis, attention-deficit-hyperactivity disorder (ADHD), and manic or psychotic depression and autism.

6. The method of claim 1, wherein the mental disorder is schizophrenia, schizophrenic form disorders or schizoaffective disorders.

7. The method of claim 1, wherein the associated disorder is selected from the group comprising a major depressive disorder, a bipolar disorder, a personality disorder, an obsessive compulsive disorder, autism, cardiovascular disorder and diabetes.

8. The method of claim 7, wherein the personality disorder is selected from the group comprising paranoid, schizoid, schizotype, antisocial and borderline disorders.

9. The method of claim 1, wherein the presence of GAG trinucleotide repeat (TNR) polymorphism in the 5′-untranslated region of the glutamate cystein ligase catalytic subunit (GCLC) gene in said biological sample is determined by Polymerase Chain Reaction and gel separation of the polymorphic fragments.

10. The method of claim 1, wherein the said biological sample is selected from the group comprising whole blood, semen, saliva, tears, urine, fecal material, sweat, buccal smears, skin, and biopsies of muscle, brain tissue, nerve tissue and hair.

11. The method of claim 1, wherein the biological sample is cultured fibroblasts extracted from skin.

12. The method of claim 2, wherein the second gene involved in an epistatic interaction with GCLC is selected from the group of genes coding for Catechol O-methyltransferase, Dysbindin, Neuregulin-1, Protein kinase B, Disrupted in schizophrenia 1 protein, glutamic acid decarboxylase 1, Regulator of G-protein signaling 4, Receptor tyrosine-protein kinase erbB-4, Serine/threonine-protein phosphatase 2B catalytic subunit gamma isoform, Early growth response protein 3, Proline dehydrogenase 2, D-amino acid oxidase activator, Neuronal acetylcholine receptor subunit alpha-2, Glutamate-cysteine ligase modifier subunit, Glutathione synthetase, Glutathione peroxidase 1, Gamma-glutamyltranspeptidase 1, Nuclear respiratory factor 1, Nuclear factor erythroid 2-related factor or Nuclear factor erythroid 2-related factor 3.

13. A kit for predicting susceptibility to a mental disorder, or a mental associated disorder, in a subject comprising
i) at least one primer and/or probe for determining the presence of GAG trinucleotide repeat (TNR) polymorphism in the 5′-untranslated region of the glutamate cystein ligase catalytic subunit (GCLC) gene in a biological sample, wherein said GAG trinucleotide repeat (TNR) polymorphism is associated with a mental disorder, or a mental associated disorder,
ii) and optionally with reagents and/or instructions for use.

14. The kit of claim 13 further comprising at least one primer and/or probe for determining the presence of at least one polymorphism in a second gene involved in an epistatic interaction with GCLC.

15. The kit of claim 14, wherein the second gene involved in an epistatic interaction with GCLC is selected from the, group of genes coding for Catechol O-methyltransferase, Dysbindin, Neuregulin-1, Protein kinase B, Disrupted in schizophrenia 1 protein, glutamic acid decarboxylase 1, Regulator of G-protein signaling 4, Receptor tyrosine-protein kinase erbB-4, Serine/threonine-protein phosphatase 2B catalytic subunit gamma isoform, Early growth response protein 3, Proline dehydrogenase 2, D-amino acid oxidase activator, Neuronal acetylcholine receptor subunit alpha-2, Glutamate-cysteine ligase modifier subunit, Glutathione synthetase, Glutathione peroxidase 1, Gamma-glutamyltranspeptidase 1, Nuclear respiratory factor 1, Nuclear factor erythroid 2-related factor or Nuclear factor erythroid 2-related factor 3.

16. A prognostic composition for predicting susceptibility to a mental disorder, or a mental associated disorder, in a subject comprising
i) at least one primer and/or probe for determining the presence of GAG trinucleotide repeat (TNR) polymorphism in the 5′-untranslated region of the glutamate cystein ligase catalytic subunit (GCLC) gene in a biological sample, wherein said GAG trinucleotide repeat (TNR) polymorphism is associated with a mental disorder, or a mental associated disorder,
ii) and optionally with reagents and/or instructions for use.

17. The prognostic composition of claim 16 further comprising at least one primer and/or probe for determining the presence of at least one polymorphism in a second gene involved in an epistatic interaction with GCLC.

18. The prognostic composition of claim 17, wherein the second gene involved in an epistatic interaction with GCLC is selected from the group of genes coding for Catechol O-methyltransferase, Dysbindin, Neuregulin-1, Protein kinase B, Disrupted in schizophrenia 1 protein, glutamic acid decarboxylase 1, Regulator of G-protein signaling 4, Receptor tyrosine-protein kinase erbB-4, Serine/threonine-protein phosphatase 2B catalytic subunit gamma isoform, Early growth response protein 3, Proline dehydrogenase 2, D-amino acid oxidase activator, Neuronal acetylcholine receptor subunit alpha-2, Glutamate-cysteine ligase modifier subunit, Glutathione synthetase, Glutathione peroxidase 1, Gamma-glutamyltranspeptidase 1, Nuclear respiratory factor 1, Nuclear factor erythroid 2-related factor or. Nuclear factor erythroid 2-related factor 3.

19. Use of a primer and/or probe for determining the presence of GAG TNR polymorphism in the 5′-untranslated region of the GCLC gene in the manufacture of a prognostic composition.

20.-30. (canceled)
